(19) United States
(12) Patent Application Publication
(10) Pub. No.: US 2026/0294263 A1
(43) Pub. Date: Oct. 1, 2026

(54) Methods for Predicting Atrial Fibrillation (AFIB) Occurrences Using a Wearable Device

(71) Applicant: Google LLC, Mountain View, CA (US)

(72) Inventors: Zeinab Esmaeilpour, San Francisco, CA (US); Anthony Zahi Faranesh, Oakland, CA (US)

(21) Appl. No.: 19/477,390

(22) PCT Filed: Apr. 21, 2023
(86) PCT No.: PCT/US2023/019397

§ 371 Date: Oct. 21, 2025

(51) Int. Cl. A61B 5/024; A61B 5/00

(52) U.S. Cl. CPC A61B 5/02438; A61B 5/02416; A61B 5/681; A61B 5/7264; A61B 5/7275; A61B 5/7282

(57) ABSTRACT

A method for predicting a cardiac arrhythmia event is provided. The method includes determining a user wearing a wearable computing device has experienced an initial cardiac arrhythmia event based, at least in part, on biometric data obtained from one or more biometric sensors of the wearable computing device. The method includes obtaining biometric data indicative of a cardiac rhythm of the user for an observation period in response to determining the user has experienced the initial cardiac arrhythmia event. The method includes determining one or more temporal patterns of cardiac arrhythmia events of the user during the observation period. The method includes generating a prediction for at least one future cardiac arrhythmia event based, at least in part, on the one or more temporal patterns of cardiac arrhythmia events of the user during the observation period.

### PRIORITY CLAIMS

The present application is based on and claims priority to International Patent Application Serial No. PCT/US2023/019397, having a filing date of Apr. 23, 2023, which is incorporated herein by reference.

### FIELD

Example aspects of the present disclosure relate generally to wearable computing devices. More particularly, the present disclosure relates to methods for predicting a cardiac arrhythmia event of a user of the wearable computing device.

### BACKGROUND

A wearable computing device can be worn, for instance, on a user's wrist. The wearable computing device can include a plurality of sensors such as, e.g., biometric sensors. The biometric sensors can obtain data indicative of a cardiac rhythm of the user.

### SUMMARY

Aspects and advantages of embodiments of the present disclosure will be set forth in part in the following description, or can be learned from the description, or can be learned through practice of the embodiments.

In one aspect, a method for predicting a cardiac arrhythmia event is provided. The method includes determining a user wearing a wearable computing device has experienced an initial cardiac arrhythmia event based, at least in part, on biometric data obtained from one or more biometric sensors of the wearable computing device. The method includes, responsive to determining the user has experienced the initial cardiac arrhythmia event, obtaining biometric data indicative of a cardiac rhythm of the user for an observation period. The method includes determining one or more temporal patterns of cardiac arrhythmia events of the user during the observation period. The method includes generating a prediction for at least one future cardiac arrhythmia event based, at least in part, on the one or more temporal patterns of cardiac arrhythmia events of the user during the observation period. Generating a prediction for at least one future cardiac arrhythmia event may relate to generating at least one prediction signal indicating whether at least one future cardiac arrhythmia event will occur during a predetermined prediction period.

In some implementations, the initial cardiac arrhythmia event includes an atrial fibrillation (afib) event.

In some implementations, obtaining the biometric data indicative of a cardiac rhythm of the user includes collecting biometric data indicative of a cardiac rhythm of the user for a duration of the observation and generating a probability model based, at least in part, on the biometric data indicative of the cardiac rhythm of the user.

In some implementations, generating the prediction for the at least one future cardiac arrhythmia event includes generating at least one prediction signal indicative of the future cardiac arrhythmia event.

In some implementations, generating the prediction for the at least one future cardiac arrhythmia event includes providing the probability model as an input to a predictive model. In some implementations, the predictive model is configured to predict the future cardiac arrhythmia event based, at least in part, on the probability model and a classifier.

In some implementations, the probability model is a transition matrix including a plurality of probability values that are determined, based at least in part, on the biometric data indicative of the cardiac rhythm of the user. In such implementations, the transition matrix includes a first probability value, a second probability value, a third probability value, and a fourth probability value.

In some implementations, the observation period includes a plurality of sampling periods. Furthermore, in such implementations, the first probability value is indicative of a probability of the user remaining in a first cardiac state in consecutive sampling period, the second probability value is indicative of a probability of the user transitioning from the first cardiac state to a second cardiac state in consecutive sampling periods, the third probability value is indicative of a probability of the user transitioning from the second cardiac state to the first cardiac state in consecutive sampling periods, and the fourth probability value is indicative of a probability of the suer remaining in the second cardiac state in consecutive sampling periods. In some implementations, the first cardiac state is indicative of a sinus rhythm, and the second cardiac state is indicative of a cardiac arrhythmia.

In some implementations, the one or more temporal patterns of cardiac arrhythmia events of the user are monitored, based at least in part, on the biometric data indicative of the cardiac rhythm of the user obtained during the observation period.

In some implementations, a duration of the observation period is based, at least in part, on the initial cardiac arrhythmia.

In some implementations, the method can include transmitting a recommendation to the user to wear an electrocardiogram (ECG) monitor in response to generating the prediction of the at least one future cardiac arrhythmia event. In some implementations, the recommendation can be a recommendation signal indicative of the recommendation to the user to wear the ECG monitor.

In some implementations, the method can include transmitting the at least one prediction signal indicative of the future cardiac arrhythmia event. In some implementations, transmitting the at least one prediction signal includes transmitting the at least one prediction signal to the user of the wearable computing device. Furthermore, the user can be alerted to the at least one prediction signal via a notification on the wearable computing device.

In another aspect, a wearable computing device is provided. The wearable computing device includes one or more sensors configured to obtain biometric data of a user wearing the wearable computing device. The wearable computing device includes one or more computing devices. The one or more computing devices are configured to determine the user has experienced an initial cardiac arrhythmia event. The one or more computing devices are configured to obtain biometric data indicative of a cardiac rhythm of the user for an observation period in response to determining the user has experienced the initial cardiac arrhythmia event. The one or more computing devices are configured to determine one or more temporal patterns of cardiac arrhythmia events of the user during the observation period. The one or more computing devices are configured to generate a prediction for a future cardiac arrhythmia event based, at least in part, on the one or more temporal patterns of cardiac arrhythmia events of the user during the observation period.

In some implementations, the initial cardiac arrhythmia event includes an atrial fibrillation (afib) event.

In some implementations, the one or more sensors include a photoplethysmogram (PPG) sensor. In such implementations, the biometric data indicative of the cardiac rhythm of the user includes a PPG signal.

In some implementations, the one or more computing devices are further configured to collect the biometric data indicative of the cardiac rhythm of the user for a duration of the observation period and generate a probability model based, at least in part, on the biometric data indicative of the cardiac rhythm of the user.

In some implementations, the observation period includes a plurality of sampling periods. Furthermore, in some implementations, the probability model is a transition matrix including a first probability value indicative of a probability of the user remaining in a first cardiac state in consecutive sampling period, a second probability value indicative of a probability of the user transitioning from the first cardiac state to a second cardiac state in consecutive sampling periods, a third probability value indicative of a probability of the user transitioning from the second cardiac state to the first cardiac state in consecutive sampling periods, and a fourth probability value indicative of a probability of the user remaining in the second cardiac state in consecutive sampling periods.

In some implementations, the one or more computing devices are further configured to provide the probability model as an input to a predictive model. In some implementations, the predictive model is configured to predict the future cardiac arrhythmia event based, at least in part, on the probability model and a classifier.

In some implementations, the one or more computing devices are configured to receive a signal from the predictive model indicative of a prediction of the future cardiac arrhythmia event. The one or more computing devices are further configured to generate at least one prediction signal based, at least in part, on the signal received from the predictive model.

In some implementations, a duration of the observation period is based, at least in part, on the initial cardiac arrhythmia event.

In another aspect, a non-transitory computer-readable medium storing instructions is provided. The non-transitory computer-readable medium stores instructions that, when executed by one or more processors of a computing device, cause the one or more processors to perform operations. The operations include determining a user wearing a wearable computing device has experienced an initial cardiac arrhythmia event based, at least in part, on biometric data obtained from one or more biometric sensors of the wearable computing device. The operations include, responsive to determining the user has experienced the initial cardiac arrhythmia event, obtaining biometric data indicative of a cardiac rhythm of the user for an observation period. The operations include determining one or more temporal patterns of cardiac arrhythmia events of the user during the observation period. The operations include generating a prediction for at least one future cardiac arrhythmia event based, at least in part, on the one or more temporal patterns of cardiac arrhythmia events of the user during the observation period.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1 depicts an embodiment of a wearable computing device according to example embodiments of the present disclosure;

FIG. 2 depicts a front perspective view of a wearable computing device according to example embodiments of the present disclosure;

FIG. 3 depicts a rear perspective view of the wearable computing device of FIG. 3 according to example embodiments of the present disclosure;

FIG. 4 depicts a block diagram of components of a wearable computing device according to example embodiments of the present disclosure;

FIG. 5 depicts a block diagram of a machine-learned model for predicting a future cardiac arrhythmia event for a user of a wearable computing device according to example embodiments of the present disclosure;

FIG. 6 depicts an example method for predicting a cardiac arrhythmia event according to example embodiments of the present disclosure;

FIG. 7 depicts a correlated plot and probability model according to example embodiments of the present disclosure; and

FIG. 8 depicts a computing system according to example embodiments of the present disclosure.

Repeat use of reference characters in the present specification and drawings is intended to represent the same and/or analogous features or elements of the present invention.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Example aspects of the present disclosure are directed to predicting future cardiac arrhythmia events (e.g., atrial fibrillation (afib) events) of a user. For instance, an atrial fibrillation event can be detected based on data indicative of a cardiac rhythm of the user that is obtained by sensors onboard a wearable computing device (e.g., a smartwatch) worn by the user. In some embodiments, the wearable computing device can be configured to notify the user of the detected atrial fibrillation event and recommend further testing and/or treatments. For instance, the wearable computing device can recommend the user begin monitoring their cardiac rhythm with an electrocardiogram (ECG), which is the method for diagnosis most preferred by doctors and clinicians. However, due to the paroxysmal (sporadic) and sometimes asymptomatic nature of arrhythmias such as atrial fibrillation, monitoring with short duration ECGs can often miss detection.

Example aspects of the present disclosure are directed to predicting future atrial fibrillation events of a user based on historical cardiac rhythm data obtained from sensors onboard a wearable computing device worn by a user. For instance, in some implementations, the wearable computing device can include an optical sensor (e.g., photoplethysmogram (PPG) sensor) configured to obtain biometric data indicative of a cardiac rhythm of the user. In such implementations, the biometric data can include a PPG signal that can, for instance, be indicative of an initial atrial fibrillation event of the user.

In some implementations, the wearable computing device can be configured to obtain further biometric data of the user for the duration of an observation period in response to detecting the initial atrial fibrillation event. For instance, the wearable computing device can be configured to monitor temporal patterns of the biometric data during the observation period by producing a binary value for each sampling period (e.g., 24 hours) of the observation period. The binary value can be indicative of whether an atrial fibrillation event was detected during that respective sampling period. For example, a binary value of 0 can indicate that no atrial fibrillation event was detected during a respective sampling period, whereas a binary value of 1 can indicate that an atrial fibrillation event was detected during the respective sampling period. Put differently, in response to determining whether an atrial fibrillation event was detected, the wearable computing device can be configured to store one of two binary values (i.e., 0 indicating no detected atrial fibrillation event, 1 indicating a detected atrial fibrillation event).

In some implementations, a probability model can be generated based, at least in part, on the binary values. Furthermore, in some implementations, the probability model can include a transition probability matrix. The transition probability matrix can include a plurality of probability values indicative of the probability of the user transitioning between cardiac rhythm states (e.g., sinus rhythm, atrial fibrillation) in consecutive sampling periods of the observation period. By way of example, the transition probability matrix can include a first probability value indicative of the probability the user will remain in a sinus rhythm in consecutive sampling periods, a second probability value can be indicative of the probability the user will transition from a sinus rhythm to atrial fibrillation in consecutive sampling periods, a third probability value can be indicative of the probability the user will transition from atrial fibrillation to a sinus rhythm in consecutive sampling periods, and a fourth probability value can be indicative of the user remaining in atrial fibrillation in consecutive sampling periods.

According to example aspects of the present disclosure, the transition probability matrix can be used as input to a machine learning model configured to predict a future atrial fibrillation event of the user. For instance, the transition probability matrix can be used as a feature to predict whether a future atrial fibrillation event will occur during a prediction period (e.g., two weeks). More specifically, at least one prediction signal indicative of the future atrial fibrillation event within the prediction period can be generated. In this way, example aspects of the present disclosure can be used by the user to determine likelihood of the future atrial fibrillation event being detected by a clinical measurement device, such as an ECG patch, during the prediction period, based at least in part on the at least one prediction signal indicative of the future atrial fibrillation event.

A wearable computing device according to example aspects of the present disclosure provide numerous technical effects and benefits. For instance, as noted above, atrial fibrillation is frequently asymptomatic and, as such, is often not reliably identifiable. Thus, example aspects of the present disclosure provide a method for predicting when a future atrial fibrillation event will occur in order to optimize detection of atrial fibrillation in the user by a clinical measurement device, such as an ECG patch. In this way, example aspects of the present disclosure provide a data-driven non-invasive method to optimize detection of atrial fibrillation in the user. Furthermore, example aspects of the present disclosure can decrease the chances of a false positive and/or misdiagnoses of atrial fibrillation after an initial atrial fibrillation event is detected. In this way, example aspects of the present disclosure can lead to proper diagnosis and more effective treatment of the user.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (e.g., “A or B” is intended to mean “A or B or both”). The term “at least one of” in the context of, e.g., “at least one of A, B, and C” refers to only A, only B, only C, or any combination of A, B, and C. In addition, here and throughout the specification and claims, range limitations may be combined and/or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a 10 percent margin, i.e., including values within ten percent greater or less than the stated value. In this regard, for example, when used in the context of an angle or direction, such terms include within ten degrees greater or less than the stated angle or direction, e.g., “generally vertical” includes forming an angle of up to ten degrees in any direction, e.g., clockwise or counterclockwise, with the vertical direction V.

The word “exemplary” is used herein to mean “serving as an example, instance, or illustration.” In addition, references to “an embodiment” or “one embodiment” does not necessarily refer to the same embodiment, although it may. Any implementation described herein as “exemplary” or “an embodiment” is not necessarily to be construed as preferred or advantageous over other implementations. Moreover, each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents.

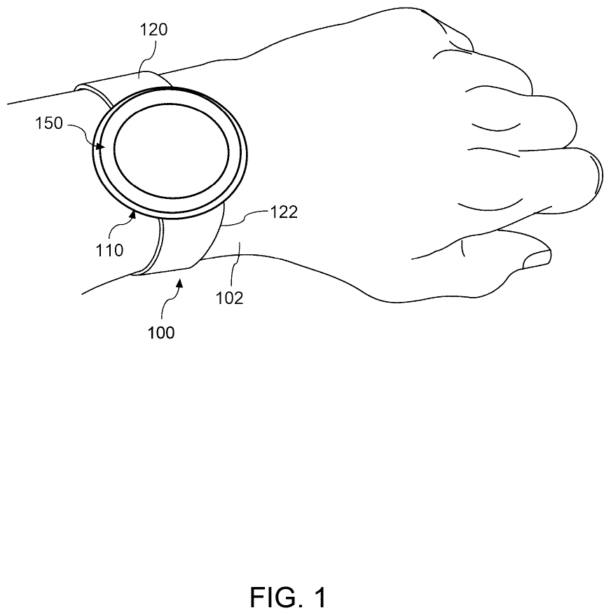

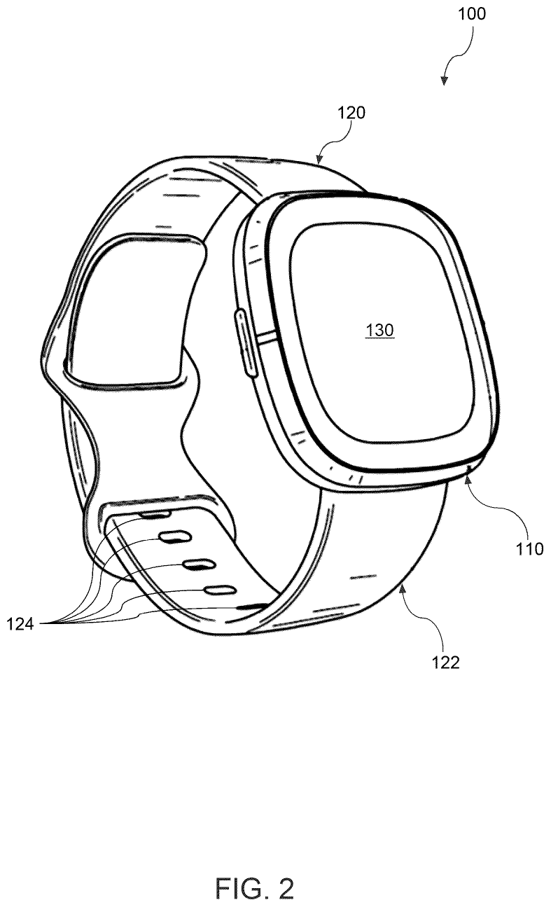

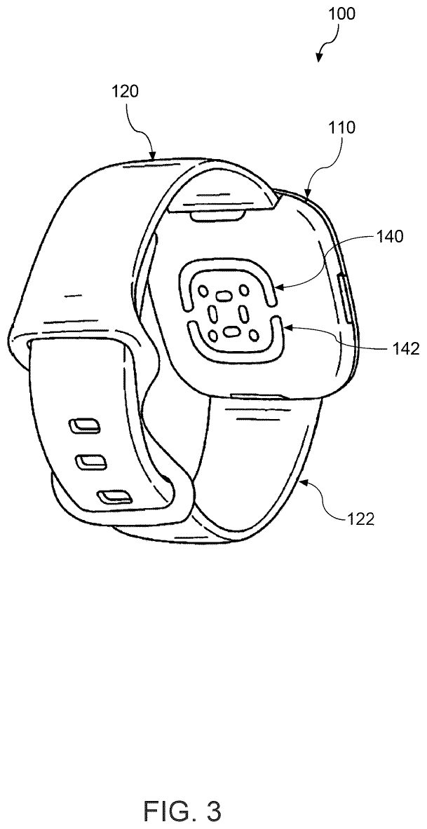

Referring now to the Figures, FIG. 1 through FIG. 3 illustrate a wearable computing device 100 according to some implementations of the present disclosure. As shown, the wearable computing device 100 can be worn, for instance, on an arm 102 (e.g., wrist) of a user. The wearable computing device 100 can include a housing 110. The housing 110 can define a cavity (e.g., internal volume) (not shown) in which one or more electronic components (e.g., disposed on printed circuit boards) are disposed. For instance, the wearable computing device 100 can include a printed circuit board (e.g., flexible printed circuit board) (not shown) disposed within the cavity. Furthermore, one or more electronic components can be disposed on the printed circuit board. The wearable computing device 100 can further include a battery (not shown) that is disposed within the cavity defined by the housing.

As shown, the wearable computing device 100 can include a first band 120 coupled to the housing 110 at a first location and a second band 122 coupled to the housing 110 at a second location. The first band 120 and the second band 122 can be coupled to one another at a third location (not shown) to secure the housing 110 to the arm 102 of the user. For instance, the first band 120 can include a buckle or clasp (not shown). Additionally, the second band 122 can define a plurality of apertures 124 spaced apart from one another along a length of the second band 122. In such embodiments, a prong of the buckle associated with the first band 120 can extend through one of the plurality of openings defined by the second band 122 to couple the first band 120 to the second band 122.

It should be appreciated that the first band 120 can be coupled to the second band 122 using any suitable type of fastener. For instance, in some embodiments, the first band 120 and the second band 122 can include a magnet (not shown). In such embodiments, the first band 120 and the second band 122 can be magnetically coupled to one another to secure the housing 110 to the arm 102 of the user.

The wearable computing device 100 can include a display 130 configured to display content (e.g., time, date, biometric, notifications, etc.) for viewing by the user. The display 130 can include a plurality of pixels. For instance, in some embodiments, the display 130 can include an organic light-emitting diode (OLED) display. It should be understood, however, that the display 130 can include any suitable type of display.

In some implementations, the wearable computing device 100 can include a first electrode 140 and a second electrode 142. It should be understood that the wearable computing device 100 can include more or fewer electrodes. As shown, the first electrode 140 and the second electrode 142 are positioned with respective apertures (e.g., cutouts) defined by the housing 110. Furthermore, the first electrode 140 and the second electrode 142 can each contact (e.g., touch) the wrist of the user. In this manner, the first electrode 140 and the second electrode 142 can be used to measure one or more biometrics (e.g., electrodermal activity, electrocardiogram) of the user.

The wearable computing device 100 can include a display cover 150 positioned on the housing 110 such that the display cover 150 is positioned on top of the display 130. In this manner, the display cover 150 can protect the display 130 from being damaged (e.g., scratched or cracked). In some embodiments, the wearable computing device 100 can include a seal (not shown) positioned between the housing 110 and the display cover 150. For instance, a first surface of the seal can contact the housing 110 and a second surface of the seal can contact the display cover 150. In this manner, the seal between the housing 110 and the display cover 150 can prevent a liquid (e.g., water) from entering the cavity defined by the housing 110.

It should be understood that the display cover 150 can be optically transparent so that the user can view information being displayed on the display 130. For instance, in some embodiments, the display cover 150 can include a glass material. It should be understood, however, that the display cover 150 can include any suitable optically transparent material.

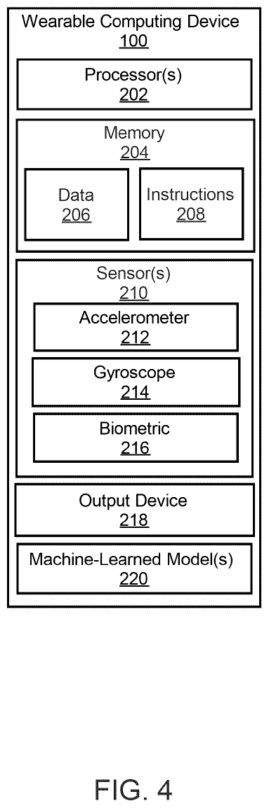

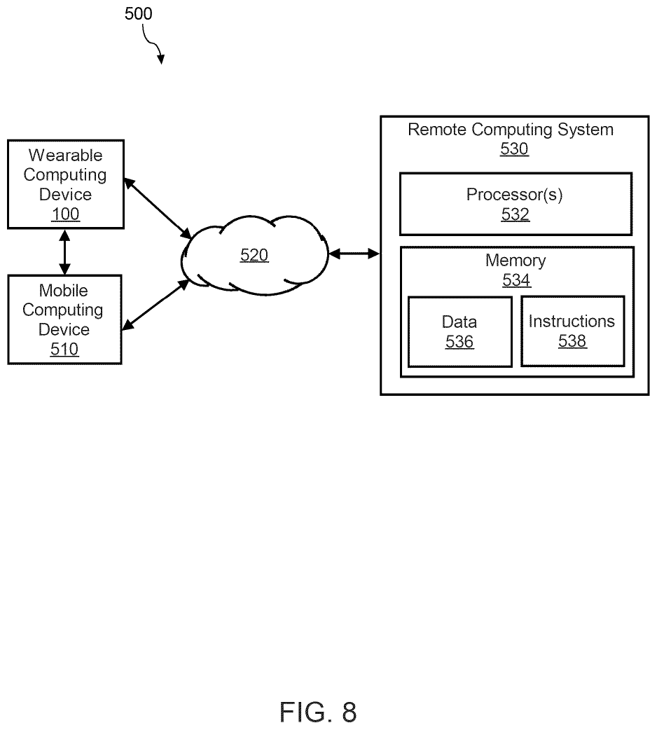

Referring now to FIG. 4, a block diagram of components of a wearable computing device 100 is provided according to some embodiments of the present disclosure. It should be understood that the wearable computing device 100 can be implemented within the system 500 discussed below with reference to FIG. 8.

As shown, the wearable computing device 100 can include one or more processors 202. The one or more processors 202 can include any suitable processing device (e.g., a processor core, a microprocessor, an application specific integrated circuit (AISC), a field programmable gate array (FPGA), a microcontroller, etc.). The wearable computing device 100 can further include a memory 204. The memory 204 can include one or more non-transitory computer-readable storage media, such as random access memory (RAM), read-only memory (ROM), electronically erasable programmable ready-only memory (EEPROM), erasable programmable read-only memory (EPROM), flash memory devices, and combinations thereof. The memory 204 can store data 206 and instructions 208 that, when executed by the one or more processors 202, cause the one or more processors 202 to perform operations disclosed herein.

The wearable computing device 100 can include a plurality of sensors 210. For instance, in some embodiments, the plurality of sensors 210 can include an accelerometer 212 (e.g., a multi-axis accelerometer) and a gyroscope 214. In this manner, the accelerometer 212, the gyroscope 214, or both can obtain motion data (e.g., acceleration, angular velocity) indicative of movement of the user.

In some embodiments, the plurality of sensors 210 can include one or more biometric sensors 216. For instance, in some embodiments, the one or more biometric sensors 216 can include a photoplethysmogram (PPG) sensor. Alternatively, or additionally, the sensors 210 can include a temperature sensor (not shown) configured to detect a body temperature of the user wearing the wearable computing device 100.

In some embodiments, the wearable computing device 100 can include one or more output devices 218. For instance, the one or more output devices 218 can include a display screen (e.g., display 130). In this manner, the wearable computing device 100 can display content (e.g., notifications) that can be viewed by the user. Alternatively, or additionally, the one or more output devices 218 can include one or more speakers. In this manner, the wearable computing device 100 can emit audible noises (e.g., alarm, voice automated message, etc.) for the user. As will be discussed below, the wearable computing device 100 can be configured to assess a future cardiac arrhythmia risk of the user.

In some embodiments, the one or more processors 202 can be communicatively coupled to the plurality of sensors 210. For instance, the one or more processors 202 can be communicatively coupled to the plurality of sensors 210 via a data interface (e.g., data bus). In this manner, the one or more processors 202 can obtain data from the plurality of sensors 210. In some embodiments, the one or more processors 202 can determine a future cardiac arrhythmia event of the user based, at least in part, on the data obtained from the one or more sensors 210.

In some embodiments, biometric data obtained from the one or more biometric sensors 216 of the wearable computing device 100 can indicate whether the wearable computing device 100 is currently being worn by the user. For instance, in some embodiments, the biometric data obtained from the one or more biometric sensors 216 can indicate the wearable computing device 100 is not being worn (e.g., off-wrist) by the user. In such embodiments, the one or more processors 202 can be configured to disable cardiac arrhythmia-related data collection functionality while the biometric data obtained from the one or more biometric sensors 216 indicates the wearable computing device 100 is not being worn by the user. In this manner, erroneous data from the one or more biometric sensors 216 can be ignored. It should be understood that the one or more processors 202 can be configured to enable cardiac arrhythmia-related data collection functionality when the biometric data obtained from the one or more biometric sensors 216 indicates the wearable computing device 100 is being worn (e.g., on-wrist) by the user.

In some embodiments, the wearable computing device 100 can include one or more machine-learned models 220. For instance, in some embodiments, the one or more machine-learned models 220 can be stored in the memory 204 of the wearable computing device 100. In alternative embodiments, the one or more machine-learned models 220 can be stored in the memory of one or more devices that are remote relative to the wearable computing device 100. For instance, in some embodiments, the one or more machine-learned models 220 can be stored in memory of the mobile computing device 510 (FIG. 8) that is communicatively coupled with the wearable computing device 100 via a network 520 (FIG. 8). Alternatively, or additionally, the one or more machine-learned models 220 can be stored on one or more servers (not shown) that are communicatively coupled with the wearable computing device 100 via the network 520. As will now be discussed, the one or more machine-learned models 220 can be configured to generate a future cardiac arrhythmia event prediction based, at least in part, on data obtained from the one or more sensors 210.

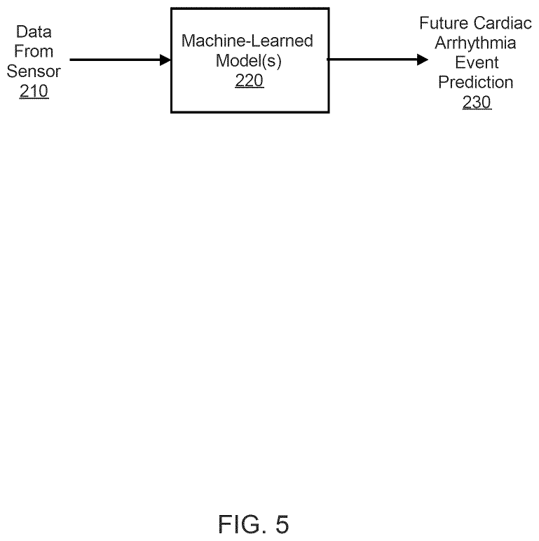

Referring now to FIG. 5, data from one or more of the sensors 210 can be provided as an input to the one or more machine-learned models 220. For instance, in some embodiments, data from at least one of the accelerometer 212 (FIG. 4), the gyroscope 214 (FIG. 4), or the one or more biometric sensors 216 (FIG. 4) can be provided as an input to the one or more machine-learned models 220. The one or more machine-learned models 220 can process the data from the one or more sensors 210 to generate a future cardiac arrhythmia event prediction based, at least in part, on data obtained from the one or more sensors 210. In some embodiments, the output of the one or more machine-learned models 220 can be a single numerical value that can be used to indicate whether a cardiac arrhythmia event will occur in the future. In alternative embodiments, the output of the one or more machine-learned models 220 can include a plurality of outputs.

It should be understood that the one or more machine-learned models 220 can include any suitable type of machine-learned model. For instance, the one or more machine-learned models 220 can include, without limitation, a convolutional neural network, a decision tree, a Bayesian network, a support vector machine, a K-means cluster, or any other suitable type of machine-learned model and/or deep learning models.

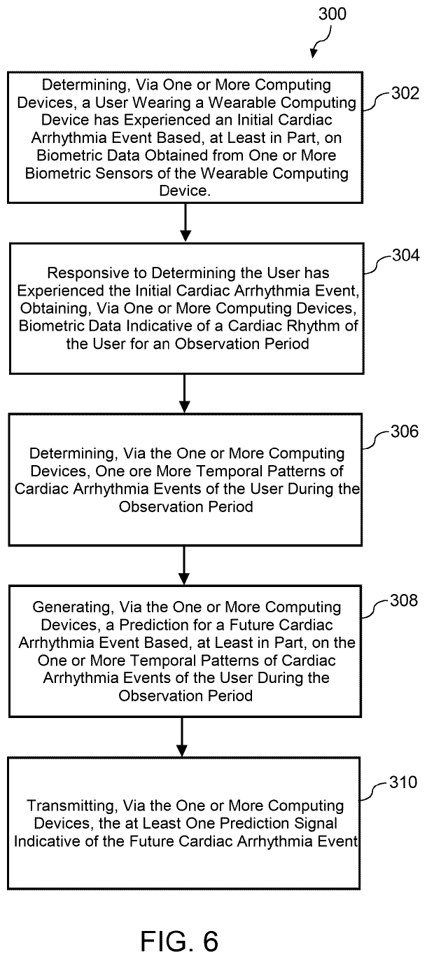

Referring now to FIG. 6, a flow diagram of a method 300 for predicting a cardiac arrhythmia event of a user of a wearable computing device is provided according to example embodiments of the present disclosure. The method 300 may be implemented using, for instance, the wearable computing device 100 discussed above with reference to FIGS. 1-5. Alternatively, the method 300 may be implemented by a computing device (e.g., server, smartphone, etc.) that is communicatively coupled to the wearable computing device 100. It should be understood that, in some embodiments, some steps of the method 300 may be implemented locally on the wearable computing device 100, whereas other steps of the method 300 may be implemented by a computing device that is remote from the wearable computing device 100 and is communicatively coupled to the wearable computing device 100 via one or more wireless networks. FIG. 6 depicts steps performed in a particular order for purposes of illustration and discussion. Those of ordinary skill in the art, using the disclosures provided herein, will understand that various steps of any of the methods described herein can be omitted, expanded, performed simultaneously, rearranged, and/or modified in various ways without deviating from the scope of the present disclosure. Furthermore, various steps (not illustrated) can be performed without deviating from the scope of the present disclosure. Additionally, the method 300 is generally discussed with reference to the wearable computing device described above with reference to FIGS. 1-5, the plurality of sensors 210 described above with reference to FIGS. 4-5, and the machine-learned models 220 described above with reference to FIGS. 4-5. However, it should be understood that aspects of the present method 300 can find application with any suitable wearable computing device, sensor, and/or machine-learned model.

At (302), the method 300 can include determining, via one or more computing devices, a user wearing a wearable computing device has experienced an initial cardiac arrhythmia event based, at least in part, on biometric data obtained from one or more biometric sensors of the wearable computing device. More particularly, one or more computing devices can be configured to determine a user wearing the wearable computing device (e.g., wearable computing device 100) has experienced an initial cardiac arrhythmia event based, at least in part, on biometric data obtained from the one or more biometric sensors (e.g., biometric sensors 216) of the wearable computing device. As noted above, in some embodiments, the one or more computing devices can be local to the wearable computing device. Alternatively, in other embodiments, the one or more computing devices can be remote to the wearable computing device and can be communicatively coupled to the wearable computing device. Furthermore, in some embodiments, the initial cardiac arrhythmia event can include an atrial fibrillation (afib) event. However, those of ordinary skill in the art will understand that the initial cardiac arrhythmia event can include any suitable cardiac arrhythmia event without deviating from the scope of the present disclosure.

Responsive to determining the user has experienced an initial cardiac arrhythmia event at (302), the method 300 can include, at (304), obtaining, via the one or more computing devices, biometric data indicative of a cardiac rhythm of the user for an observation period. More particularly, the one or more computing devices can be configured to collect biometric data indicative of a cardiac rhythm of the user from the one or more biometric sensors (e.g., biometric sensors 216) for a duration of an observation period in response to determining the user has experienced the initial cardiac arrhythmia event at (302). In some embodiments, the duration of the observation period can be based, at least in part, on the initial cardiac arrhythmia determined at (302). In some embodiments, the one or more biometric sensors can include a photoplethysmogram (PPG) sensor. In this way, the PPG sensors can obtain biometric data such as, e.g., PPG signals that can be indicative of the cardiac rhythm of the user wearing the wearable computing device (e.g., wearable computing device 100).

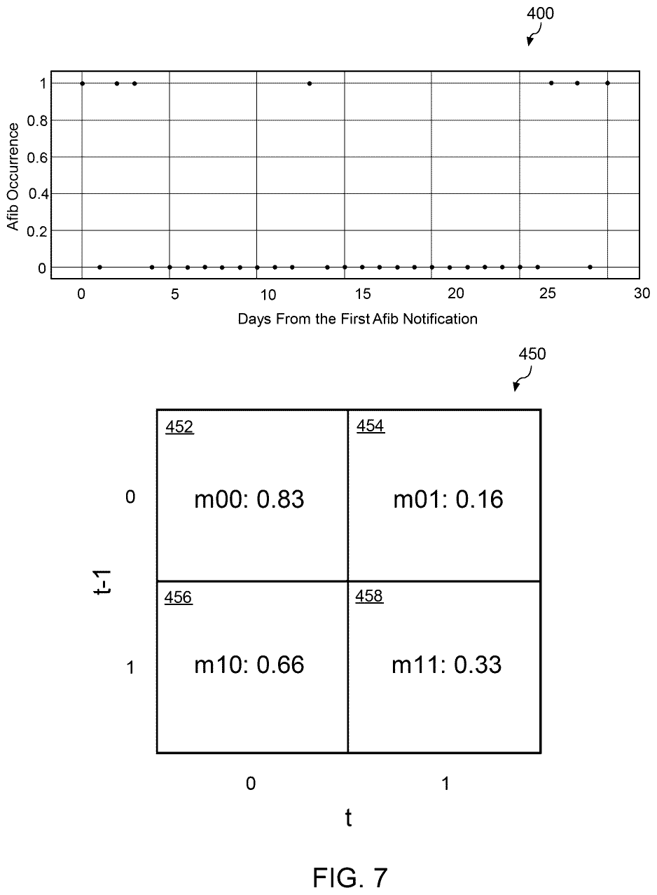

The one or more computing devices can further be configured to generate a probability model based, at least in part, on the biometric data indicative of the cardiac rhythm of the user. As will be discussed in greater detail below with reference to FIG. 7, in some embodiments, the one or more computing devices can be configured to generate a transition matrix based, at least in part, on the biometric data indicative of the cardiac rhythm of the user.

At (306), the method 300 can include determining, via the one or more computing devices, one or more temporal patterns of cardiac arrhythmia events of the user during the observation period. In some embodiments, the one or more temporal patterns of cardiac arrhythmia events of the user are monitored based, at least in part, on the biometric data indicative of the cardiac rhythm of the user obtained during the observation period. For instance, in some embodiments, the one or more temporal patterns of cardiac arrhythmia events of the user are based, at least in part, on the biometric data indicative of the cardiac rhythm of the user obtained during the observation period at (304) by the one or more biometric sensors (e.g., biometric sensors 216).

More particularly, as will be discussed in greater detail below with reference to FIG. 7, the biometric data obtained during the observation period can be used by the one or more computing devices to determine one or more temporal patterns of cardiac arrhythmia events of the user. As an illustrative example, the one or more computing devices can be configured to obtain data indicative of the cardiac rhythm of the user for a thirty-day period (e.g., observation period). During the observation period, the one or more computing devices can produce a binary value indicative of the cardiac rhythm of the user for each day of the observation period. For instance, the one or more computing devices can produce a binary value of zero when no cardiac arrhythmia events are detected for a particular sampling period (e.g., the user is in sinus rhythm). Conversely, the one or more computing devices can produce a binary value of one when a cardiac arrhythmia event is detected for a particular sampling period. Furthermore, the binary values produced over the observation period can be used in conjunction with a probability model (e.g., probability model 450) to determine the one or more temporal patterns of cardiac arrhythmia events of the user. In this way, the one or more computing devices can monitor the temporal patterns of cardiac arrhythmia events of the user during the observation period.

At (308), the method 300 can include generating, via the one or more computing devices, a prediction for at least one future cardiac arrhythmia event based, at least in part, on the one or more temporal patterns of cardiac arrhythmia events of the user during the observation period. More particularly, the one or more computing devices can generate a prediction for at least one future cardiac arrhythmia event (e.g., generate at least one prediction signal being indicative of at least one future cardiac arrhythmia event within a predetermined prediction period) based, at least in part, on the one or more temporal patterns determined at (306). In some embodiments, the prediction can be a signal generated by the one or more computing devices (e.g., machine-learned model 220). However, those of ordinary skill in the art will understand that the prediction can be of any suitable form without deviating from the scope of the present disclosure.

For instance, the one or more computing devices can provide the probability model as an input to a machine-learning model such as, e.g., a predictive model. The predictive model can be configured to predict the future cardiac arrhythmia event based, at least in part, on the probability model and a classifier. The one or more computing devices can be configured to transform the prediction into at least one prediction signal indicative of the future cardiac arrhythmia event. Those of ordinary skill in the art will understand that any suitable classifier can be used without deviating from the scope of the present disclosure.

At (310), the method 300 can include transmitting, via the one or more computing devices, the at least one prediction signal indicative of the future cardiac arrhythmia event. For example, in instances where the predictive model determines that at least one future cardiac arrhythmia event of the user will occur, the one or more computing devices can generate and communicate the at least one prediction signal to, e.g., a display of the wearable computing device informing the user (e.g., via a notification on the wearable computing device or an application on another device) that the future cardiac arrhythmia event will occur. Additionally and/or alternatively, the one or more computing devices can generate and communicate the at least one prediction signal to a third party (e.g., a physician, caretaker, etc. of the user) informing the third party that the future cardiac arrhythmia event will occur.

Furthermore, in some embodiments, the method 300 can further include transmitting, via the one or more computing devices, a recommendation to the user to wear an electrocardiogram (ECG) monitor. For instance, the one or more computing devices can transmit a recommendation signal to the user indicative of the recommendation to wear an electrocardiogram (ECG) monitor in response to generating the prediction of the at least one future cardiac arrhythmia event at (308). In some embodiments, the one or more computing devices can transmit the recommendation signal to notify the user of the recommendation via a notification on the wearable computing device or a coupled device. In this way, example aspects of the present disclosure can provide a method for alerting the user of potential future cardiac arrhythmia events based, at least in part, on biometric data obtained by the one or more biometric sensors.

Referring now to FIG. 7, as noted above, the one or more biometric sensors can be configured to collect biometric data indicative of a cardiac rhythm of the user for a duration of the observation period. In some embodiments, the one or more biometric sensors (e.g., biometric sensors 216) can collect biometric data indicative of the cardiac rhythm of the user for the duration of the observation period. For instance, plot 400 depicts exemplary biometric data obtained by the one or more biometric sensors (e.g., biometric sensors 216). The biometric data on plot 400 can be indicative of the cardiac rhythm of the user. In some embodiments, the wearable computing device 100 can be configured to collect the biometric data shown on plot 400. It should be noted that the biometric data depicted on plot 400 is for illustrative purposes only.

The observation period is depicted on the x-axis of plot 400. As noted above, the observation period can be based, at least in part, on the initial cardiac arrhythmia event of the user determined at (302). As shown, the observation period can include a plurality of sampling periods. In some embodiments, such as that shown on plot 400, the sampling period can be one day. In alternative embodiments, the sampling period can be smaller and/or larger. Those of ordinary skill in the art will understand that the sampling period can be any suitable length without deviating from the scope of the present disclosure.

Exemplary data indicative of the cardiac rhythm of the user is depicted on the y-axis of plot 400. As noted above, the one or more computing devices can be configured to produce binary values indicative of one or more cardiac states of the user during the observation period. For instance, as shown, the plot 400 can depict the one or more cardiac states of the user such as, e.g., a first cardiac state (i.e., depicted by a 0) and a second cardiac state (i.e., depicted by a 1). In some embodiments, the first cardiac state (i.e., 0) can indicate the user is in a sinus rhythm, and the second cardiac state (i.e., 1) can indicate the user is in cardiac arrhythmia. In this way, one or more temporal patterns of cardiac states of the user can be determined and monitored.

A probability model 450 can be generated based at least in part on the biometric data depicted on plot 400. As noted above, the probability model 450 can include a transition matrix. In some embodiments, the transition matrix can include a plurality of probability values. For instance, in some embodiments, the plurality of probability values can include a first probability value 452, a second probability value 454, a third probability value 456, and a fourth probability value 458. For instance, as shown in probability model 450, the first probability value 452 can be indicative of a probability of the user remaining in the first cardiac state in consecutive sampling periods. More particularly, the first probability value 452 can be indicative of a probability that the user will remain in a sinus rhythm for two consecutive sampling periods (e.g., 83% as shown on probability model 450). Furthermore, the second probability value 454 can be indicative of a probability of the user transitioning from the first cardiac state to the second cardiac state in consecutive sampling periods. More particularly, the second probability value 454 can be indicative of a probability that the user will transition from a sinus rhythm to a cardiac arrhythmia in consecutive sampling periods (e.g., 16% as shown on probability model 450). Even further, the third probability value 456 can be indicative of a probability of the user transitioning from the second cardiac state to the first cardiac state in consecutive sampling periods. More particularly, the third probability value 456 can be indicative of a probability that the user will transition from a cardiac arrhythmia to a sinus rhythm in consecutive sampling periods (e.g., 66% as shown on probability model 450). Lastly, the fourth probability value 458 can be indicative of a probability of the user remaining in a second cardiac state in consecutive sampling periods. More particularly, the fourth probability value 458 can be indicative of a probability that the user will remain in a cardiac arrhythmia in consecutive sampling periods (e.g., 33% as shown on probability model 450). As noted above, in conjunction with the cardiac rhythm data on plot 400, the probability model 450 can be used by a computing system implementing the machine-learned models described herein (e.g., computing system 500) to monitor the one or more temporal patterns of cardiac states of the user.

As an illustrative example, FIG. 7 depicts an observation period having a total of thirty sampling periods (lasting approximately twenty-four hours) on plot 400. It should be noted that, as depicted on plot 400, the first sampling period begins on Day 0 and ends on Day 1. Hence, a total of thirty transitions are used to calculate the first probability value 452, the second probability value 454, the third probability value 456, and the fourth probability value 458 shown in probability model 450.

As shown over the course of the illustrative observation period depicted in plot 400, a sinus rhythm (i.e., depicted by 0) was detected in twenty-four of the thirty sampling period. Of the twenty-four sampling periods in which a sinus rhythm was detected, a sinus rhythm was detected during the subsequent sampling period twenty times (e.g., Day 5:0, Day 6:0). Hence, when a sinus rhythm was detected during any given sampling period, the user had approximately an 83% chance of remaining in a sinus rhythm in the following sampling period (i.e., indicated by the first probability value 452 on probability model 450).

Conversely, of the twenty-four sampling periods in which a sinus rhythm was detected, a cardiac arrhythmia was detected during the subsequent sampling period four times (e.g., Day 1:0, Day 2:1). Hence, when a sinus rhythm was detected during any given sampling period, the user had approximately a 16% chance of transitioning to a cardiac arrhythmia in the following sampling period (i.e., indicated by the second probability value 454 on probability model 450).

Furthermore, as shown over the course of the illustrative observation period depicted in plot 400, a cardiac arrhythmia (i.e., depicted by 1) was detected in six of the thirty sampling periods. Of the six sampling periods in which a cardiac arrhythmia was detected, a sinus rhythm was detecting during the subsequent sampling period four times (e.g., Day 3:1, Day 4:0). Hence, when a cardiac arrhythmia was detected during any given sampling period, the user had approximately a 66% chance of transitioning to a sinus rhythm in the following sampling period (i.e., indicated by the third probability value 456 on probability model 450).

Conversely, of the six sampling periods in which a cardiac arrhythmia was detected, a cardiac arrhythmia was detecting during the subsequent sampling period two times (e.g., Day 2:1, Day 3:1). Hence, when a cardiac arrhythmia was detected during any given sampling period, the user had approximately a 33% chance of remaining in a cardiac arrhythmia in the following sampling period (i.e., indicated by the fourth probability value 458 on probability model 450).

In this way, a computing system (e.g., computing system 500) implementing the machine-learned models described herein (e.g., machine-learned models 220) can generate at least one prediction indicative of at least one future cardiac arrhythmia event based at least in part on probability values 452, 454, 456, 458. Moreover, the computing system can transform the at least one prediction into a corresponding prediction signal indicative of the at least one future cardiac arrhythmia event.

FIG. 8 depicts an example computing system 500 according to example embodiments of the present disclosure. The computing system 500 can be used, for instance, to implement the method 300 of FIG. 6 or other aspects of any of the methods described herein. The computing system 500 includes the wearable computing device 100 discussed above with reference to FIGS. 1-7 and a remote computing system 530. The wearable computing device 100 can be communicatively coupled to the server computing system 740 over a network 730.

In some embodiments, the wearable computing device 100 can communicate the data to mobile computing device 510. In such embodiments, the wearable computing device 100 can communicate the data to the mobile computing device 510 and then the mobile computing device 510 can communicate the data over the network 520 to the remote computing system 530. In alternative embodiments, the wearable computing device 100 can bypass the mobile computing device 510 and instead communicate the data directly to the remote computing system 530 via the network 520.

The remote computing system 530 includes one or more processors 532 and a memory 534. The one or more processors 532 can be any suitable processing device (e.g., a processor core, a microprocessor, an ASIC, an FPGA, a controller, a microcontroller, etc.) and can be one processor or a plurality of processors that are operatively connected. The memory 534 can include one or more non-transitory computer-readable storage medium(s), such as RAM, ROM, EEPROM, EPROM, flash memory devices, magnetic disks, etc., and combinations thereof. The memory 534 can store data 536 and instructions 538 which are executed by the processor 532 to cause the remote computing system 530 to perform operations, such as any of the operations described herein. For instance, in some embodiments, the memory 534 of the remote computing system 530 can be configured to store the one or more machine-learned models 220 discussed above with reference to FIGS. 4-5. In this manner, the data obtained from one or more sensors 310 (e.g., accelerometer, gyroscope, biometric, etc.) onboard the wearable computing device 100 and indicative of a cardiac rhythm of the user can be communicated to the remote computing system 530 and provided as an input to the one or more machine-learned models 220 stored in the memory 534 thereof. The one or more machine-learned models 220 can be configured to process the data and output a prediction for a future cardiac arrhythmia event based, at least in part, on the temporal patterns of cardiac arrhythmia events of the user during the observation period. Furthermore, the prediction of the future cardiac arrhythmia event can be communicated over the network 520 to the wearable computing device 100.

In some embodiments, the remote computing system 530 includes or is otherwise implemented by one or more computing devices. In instances in which the remote computing system 530 includes plural server computing devices, such server computing devices can operate according to sequential computing architectures, parallel computing architectures, or some combination thereof.

The network 520 can be any type of communications network, such as a local area network (e.g., intranet), wide area network (e.g., Internet), or some combination thereof and can include any number of wired or wireless links. In general, communication over the network 520 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The technology discussed herein refers to sensors and other computer-based systems, as well as actions taken, and information sent to and from such systems. One of ordinary skill in the art will recognize that the inherent flexibility of computer-based systems allows for a great variety of possible configurations, combinations, and divisions of tasks and functionality between and among components. For instance, server processes discussed herein may be implemented using a single server or multiple servers working in combination. Databases and applications may be implemented on a single system or distributed across multiple systems. Distributed components may operate sequentially or in parallel.

While the present subject matter has been described in detail with respect to specific example embodiments thereof, it will be appreciated that those skilled in the art, upon attaining an understanding of the foregoing can readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A method for predicting a cardiac arrhythmia event, the method comprising:
determining, via one or more computing devices, a user wearing a wearable computing device has experienced an initial cardiac arrhythmia event based, at least in part, on biometric data obtained from one or more biometric sensors of the wearable computing device;
responsive to determining the user has experienced the initial cardiac arrhythmia event, obtaining, via the one or more computing devices, biometric data indicative of a cardiac rhythm of the user for an observation period;
determining, via the one or more computing devices, one or more temporal patterns of cardiac arrhythmia events of the user during the observation period; and
generating, via the one or more computing devices, a prediction for at least one future cardiac arrhythmia event based, at least in part, on the one or more temporal patterns of cardiac arrhythmia events of the user during the observation period.

2. The method of claim 1, wherein the initial cardiac arrhythmia event comprises an atrial fibrillation (afib) event.

3. The method of claim 1, wherein monitoring the biometric data indicative of a cardiac rhythm of the user comprises:
collecting, via the one or more sensors, biometric data indicative of a cardiac rhythm of the user for a duration of the observation period; and
generating, via the one or more computing devices, a probability model based, at least in part, on the biometric data indicative of the cardiac rhythm of the user.

4. The method of claim 3, wherein generating the prediction for the at least one future cardiac arrhythmia event comprises providing, via the one or more computing devices, the probability model as an input to a predictive model.

5. The method of claim 4, wherein the predictive model is configured to predict the future cardiac arrhythmia event based, at least in part, on the probability model and a classifier.

6. The method of claim 3, wherein the probability model is a transition matrix comprising a plurality of probability values, the plurality of probability values comprising a first probability value, a second probability value, a third probability value, and a fourth probability value, wherein each of the plurality of probability values are determined based, at least in part, on the biometric data indicative of the cardiac rhythm of the user.

7. The method of claim 6, wherein:
the observation period comprises a plurality of sampling periods;
the first probability value is indicative of a probability of the user remaining in a first cardiac state in consecutive sampling periods;
the second probability value is indicative of a probability of the user transitioning from the first cardiac state to a second cardiac state in consecutive sampling periods;
the third probability value is indicative of a probability of the user transitioning from the second cardiac state to the first cardiac state in consecutive sampling periods; and
the fourth probability value is indicative of a probability of the user remaining in a second cardiac state in consecutive sampling periods.

8. The method of claim 7, wherein:
the first cardiac state is indicative of a sinus rhythm; and
the second cardiac state is indicative of a cardiac arrhythmia.

9. The method of claim 1, wherein the one or more temporal patterns of cardiac arrhythmia events of the user are monitored based, at least in part, on the biometric data indicative of the cardiac rhythm of the user obtained during the observation period.

10. The method of claim 1, wherein a duration of the observation period is based, at least in part, on the initial cardiac arrhythmia.

11. The method of claim 1, the method further comprising:
transmitting, via the one or more computing devices, a recommendation to the user to wear an electrocardiogram (ECG) monitor in response to generating the prediction of the at least one future cardiac arrhythmia event.

12. A wearable computing device comprising:
one or more sensors configured to obtain biometric data of a user wearing the wearable computing device; and
one or more computing devices configured to:
determine the user has experienced an initial cardiac arrhythmia event;
obtain biometric data indicative of a cardiac rhythm of the user for an observation period in response to determining the user has experienced the initial cardiac arrhythmia event;
determine one or more temporal patterns of cardiac arrhythmia events of the user during the observation period; and
generate a prediction for a future cardiac arrhythmia event based, at least in part, on the one or more temporal patterns of cardiac arrhythmia events of the user during the observation period.

13. The wearable computing device of claim 12, wherein the initial cardiac arrhythmia event comprises an atrial fibrillation (afib) event.

14. The wearable computing device of claim 12, wherein the one or more sensors comprise a photoplethysmogram (PPG) sensor, and wherein the biometric data indicative of the cardiac rhythm of the user comprises a PPG signal.

15. The wearable computing device of claim 12, wherein the one or more computing devices are further configured to:
collect, via the one or more sensors, the biometric data indicative of the cardiac rhythm of the user for a duration of the observation period; and
generate a probability model based, at least in part, on the biometric data indicative of the cardiac rhythm of the user.

16. The wearable computing device of claim 15, wherein the observation period comprises a plurality of sampling periods, and wherein the probability model is a transition matrix comprising:
a first probability value indicative of a probability of the user remaining in a first cardiac state in consecutive sampling period;
a second probability value indicative of a probability of the user transitioning from the first cardiac state to a second cardiac state in consecutive sampling periods;
a third probability value indicative of a probability of the user transitioning from the second cardiac state to the first cardiac state in consecutive sampling periods; and
a fourth probability value indicative of a probability of the user remaining in a second cardiac state in consecutive sampling periods.

17. The wearable computing device of claim 15, wherein the one or more computing devices are further configured to provide the probability model as an input to a predictive model.

18. The wearable computing device of claim 17, wherein the predictive model is configured to predict the future cardiac arrhythmia event based, at least in part, on the probability model and a classifier.

19. The wearable computing device of claim 12, wherein a duration of the observation period is based, at least in part, on the initial cardiac arrhythmia event.

20. A non-transitory computer-readable medium storing instructions that, when executed by one or more processors of a computing device, cause the one or more processors to perform operations, the operations comprising:
determining, via the computing device, a user wearing a wearable computing device has experienced an initial cardiac arrhythmia event based, at least in part, on biometric data obtained from one or more biometric sensors of the wearable computing device;
responsive to determining the user has experienced the initial cardiac arrhythmia event, obtaining, via the computing device, biometric data indicative of a cardiac rhythm of the user for an observation period;
determining, via the one or more computing devices, one or more temporal patterns of cardiac arrhythmia events of the user during the observation period; and
generating, via the computing device, a prediction for at least one future cardiac arrhythmia event based, at least in part, on the one or more temporal patterns of cardiac arrhythmia events of the user during the observation period.
